(19) United States
(12) Patent Application Publication
(10) Pub. No.: US 2024/0263142 A1
(43) Pub. Date: Aug. 8, 2024

(54) HUMAN OVARIAN CANCER NIRAPARIB-RESISTANT CELL STRAIN AND USE THEREOF

(71) Applicant: WOMEN’S HOSPITAL, ZHEJIANG UNIVERSITY SCHOOL OF MEDICINE, Hangzhou (CN)

(72) Inventors: Weiguo LU, Hangzhou (CN); Hui LIN, Hangzhou (CN); Yuqing SHEN, Hangzhou (CN); Junfen XU, Hangzhou (CN)

(21) Appl. No.: 18/625,261

(22) Filed: Apr. 3, 2024

Related U.S. Application Data
(63) Continuation of application No. PCT/CN2023/092013, filed on May 4, 2023.

(30) Foreign Application Priority Data
Jun. 29, 2022 (CN) 202210758044.5

(51) Int. Cl. C12N 5/071; C12N 5/09; G01N 33/50

(52) U.S. Cl. CPC C12N 5/0682; C12N 5/0693; G01N 33/5011; C12N 2503/02; G01N 2500/10

(57) ABSTRACT

A human ovarian cancer Niraparib-resistant cell strain and use thereof provided. A human ovarian cancer A2780 cell strain is selected as parental cell, and A2780-NiraR (preservation number: CCTCC NO: C202299) drug-resistant cell strain is established by gradually increasing concentration of PARP inhibitor Niraparib from 2 μM to 40 μM. Resistance index to Niraparib of A2780-NiraR is 8.274. In addition to resistance to Niraparib, A2780-NiraR cells are cross-resistant to Taxol. Moreover, the drug resistance of A2780-NiraR cell strain is re-tested as more than 90% of original after 3-month drug-withdrawal culture and 6-month liquid nitrogen cryopreservation and resuscitation following establishment, showing an excellent drug resistance stability. It can be used to study the tumor drug resistance mechanisms, analyze the susceptibility of chemotherapeutic drugs, screen and evaluate chemotherapeutic drugs, develop drug resistance reversal agents, and research more effective tumor treatment methods.

### CROSS-REFERENCE TO RELATED APPLICATIONS

The present application is a National Stage of International Application No. PCT/CN2023/092013 filed May 4, 2023, which claims a priority to Chinese Patent Application No. 2022107580445, filed on Jun. 29, 2022, both of which are hereby incorporated by reference in their entireties.

### TECHNICAL FIELD

The present application relates to a drug-resistant cell strain of human ovarian cancer induced by Niraparib and use thereof.

### BACKGROUND

Ovarian cancer is one of the three common malignant tumors in female reproductive organs and its lethality rate is the highest among gynecological cancers. There are about 310,000 new cases of ovarian cancer and 200,000 death cases worldwide annually. In China, there are 52,100 new cases of ovarian cancer per year, accounting for more than 15% of the global new cases; and 22,500 death cases per year, the mortality rate ranks seventh among malignant tumors in women and increases yearly, making it a serious threat to women's health. Due to its insidious symptoms, 70%-80% of patients with ovarian cancer are diagnosed in the advanced stage. In recent decades, the five-year survival rate of advanced epithelial ovarian cancer has been hovering at 30%-40% despite the improvement of diagnostic techniques and treatment methods.

In recent years, the popularity of PARP inhibitors in clinical application has revolutionized the treatment of ovarian cancer, leading to significant progress in maintenance therapy. At present, PARP inhibitors approved by FDA for maintenance treatment of platinum-sensitive recurrent ovarian cancer include Niraparib, Olaparib and Rucaparib. Breast cancer susceptibility gene (BRCA) mutation and homologous recombination deficiency (HRD) are common biomarkers in the application of PARP inhibitors. Based on the obtained clinical research evidence, FDA approved Olaparib and Rucaparib as first-line maintenance therapy for ovarian cancer patients carrying germline or somatic BRCA1/2 gene mutation with clinical complete remission or partial remission by initial chemotherapy. While Niraparib monotherapy is not limited by BRCA1/2 mutation or HRD positive, and thus has a wider applicable population compared to the other two PARP inhibitors.

Due to the unique genome mutation and copy number change in high-grade serous ovarian cancer, 50% of high-grade serous ovarian cancer shows HRD, making it highly sensitive to synergistic lethal mode. However, despite initially sensitive to PARP inhibitors, ovarian cancer patients inevitably develop acquired drug-resistance, and many patients still end up dying from ovarian cancer after exhausting treatment plans. Therefore, overcoming drug resistance is the key to cure ovarian cancer. Establishing an ovarian cancer Niraparib-resistant cell strain can provide an essential model for the research on tumor drug resistance, which has practical value.

Ovarian adenocarcinoma A2780 cell strain is derived from the tumor tissue of an untreated ovarian cancer patient, and is a very common cell strain to study ovarian cancer. At present, there are 2 main ways to induce drug resistance in vitro: the intermittent stimulation method and the gradient increasing method. However, each of the two methods has its own advantages and disadvantages. Intermittent stimulation method uses a high-dose intermittent administration mode. Although this method can better reflect the clinical situation, the final established drug-resistant strain is not highly drug-resistant. At the same time, high rate of failed induction is another problem because of the high-dose administration in the induction process. Gradient increasing method is currently a widely used method, which can effectively establish a stable and highly drug-resistant model, but it has the disadvantages of long induction period and low screening efficiency. In vitro concentration gradient increasing induction method is staged in the induction cycle and has a large span in the administration concentration, which is characterized by the formation of drug-resistant monoclonal communities, thus reducing loss rate, improving screening efficiency, is the integration of the intermittent stimulation method and the gradient increasing method. Therefore, the present application adopts the in vitro concentration gradient increasing induction method to induce the generation of drug-resistant cells.

### SUMMARY

The present application is to establish a human ovarian cancer Niraparib-resistant cell strain A2780-NiraR, and provide a drug-resistant tumor cell model for the following relevant studies: studying the morphology and biological characteristics of drug-resistant tumor cells, studying tumor multidrug resistance mechanisms, analyzing chemotherapeutic drug susceptibility, screening chemotherapeutic drugs, and researching more effective tumor treatment methods.

The present application adopts the following technical solutions:

A human ovarian cancer Niraparib-resistant cell strain was preserved in China Center for Type Culture Collection on May 18, 2022, with a preservation number of CCTCC NO: C202299. The classification name is: Human ovarian cancer Niraparib-resistant cell strain A2780-NiraR, and the preservation address is: Wuhan University, Wuhan, China.

Further, a resistance index of A2780-NiraR cell strain to Niraparib is 8.274.

Further, a drug resistance of A2780-NiraR cell strain is 91.7% of original after 3-month drug-withdrawal culture, 6-month liquid nitrogen cryopreservation and resuscitation following establishment.

The present application further provides uses of the human ovarian cancer Niraparib-resistant cell strain, including one or more of the following:
(1) study of tumor drug resistance mechanisms in vitro;
(2) analysis of chemotherapeutic drug susceptibility in vitro;
(3) preparation of a tumor cell model or a tumor animal model; where the tumor cell model includes the progeny cells established from this cell strain or the cells established by transfection with luciferase genes; the animal tumor model includes human ovarian cancer animal model established by subcutaneous tumor-bearing or tail vein injection;
(4) screening and evaluation of chemotherapeutic drugs in vitro; the method for screening tumor chemotherapeutic drugs can be as follows: adding different chemotherapeutic drugs in the culture medium of human ovarian cancer Niraparib-resistant cell strain, and observing the cytotoxicity of the drugs to obtain preliminarily effective candidate drugs; then, applying the candidate drugs to the above cells, and calculating the IC50 of the screened effective drugs; further applying the drug with the lowest IC50 to the animal model, and comparing the survival period, tumor size and metastasis status of the animals with the untreated group, so as to screen out the potential drugs to treat human ovarian cancer;
(5) development of drug resistance reversal agents in vitro; the method for developing drugs for the reversion of tumor drug resistance can be as follows: adding drug resistance reversal agents and Niraparib/Taxol in the culture medium of human ovarian cancer Niraparib-resistant cell strain, and observing the cytotoxicity of the drugs to obtain preliminarily effective candidate drugs; then, applying the candidate drugs to the above cells, and calculating the IC50 of the screened effective drugs; further applying the drug with the lowest IC50 to the animal model, and comparing the survival period, tumor size and metastasis status of the animals with the untreated group, so as to screen out the potential drugs for the reversion of drug resistance.

The present application has the beneficial effects that A2780-NiraR cells can stably grow, subculture and resuscitate in 0.05 μM Niraparib, and has the resistance index to Niraparib of 8.274. In addition to resistance to Niraparib, A2780-NiraR cells are cross-resistant to Taxol. Moreover, after the establishment of the drug-resistant cell strain, the drug resistance was re-tested after cell resuscitation following 3-month drug-withdrawal culture and 6-month liquid nitrogen cryopreservation, with a drug resistance of more than 90% of original, thus showing an excellent drug resistance stability. The present application provides a cell model for studying tumor drug resistance mechanisms, analyzing the susceptibility of chemotherapeutic drugs, screening and evaluating chemotherapeutic drugs, developing drug resistance reversal agents, and researching more effective tumor treatment methods.

### BRIEF DESCRIPTION OF DRAWINGS

FIG. 1 shows the morphological diagrams of A2780 and A2780-NiraR cells in logarithmic growth phase;

FIG. 2 shows the growth curves of A2780 and A2780-NiraR cells;

FIG. 3 shows the results of the corresponding cytotoxicity experiments of Niraparib (A) and Taxol (B) on A2780 and A2780-NiraR cells.

### DESCRIPTION OF EMBODIMENTS

The establishment steps of the drug-resistant cell strain of the present application are as follows:
(1) The human ovarian cancer cell strain A2780 was purchased from Sigma-Aldrich, USA. After resuscitation, it was cultured in a DMEM high glucose medium (containing 10 wt % fetal bovine serum) in the incubator with 5 vol % CO₂ at 37° C.
(2) A2780 cells in the logarithmic growth period were taken, the medium was replaced with fresh medium supplemented with 2 μM Niraparib, and the cells were routinely cultured in the incubator with 5 vol % CO₂ at 37° C.; after 48 h of treatment, most parental cells died, and the drug induction was stopped, a culture medium without Niraparib was used for more than one week of culture, until the drug-resistant monoclonal communities were formed and fused, and the cells resumed stable growth;
(3) the same drug induction and drug-withdrawal culture processes were repeated for 1-2 times, after the cells were in good condition, the Niraparib induction of the next increased concentration was carried out, being 5 μM, 10 μM, 20 μM and 40 μM respectively;
(4) A2780-NiraR cell strain was established in 8 months, which can stably grow, subculture and resuscitate in 0.05 μM of Niraparib.

Morphological observation and biological characteristics identification were carried out for the established A2780-NiraR cells:
1. Morphological observation: the morphology of living cells was observed by the inverted microscope.

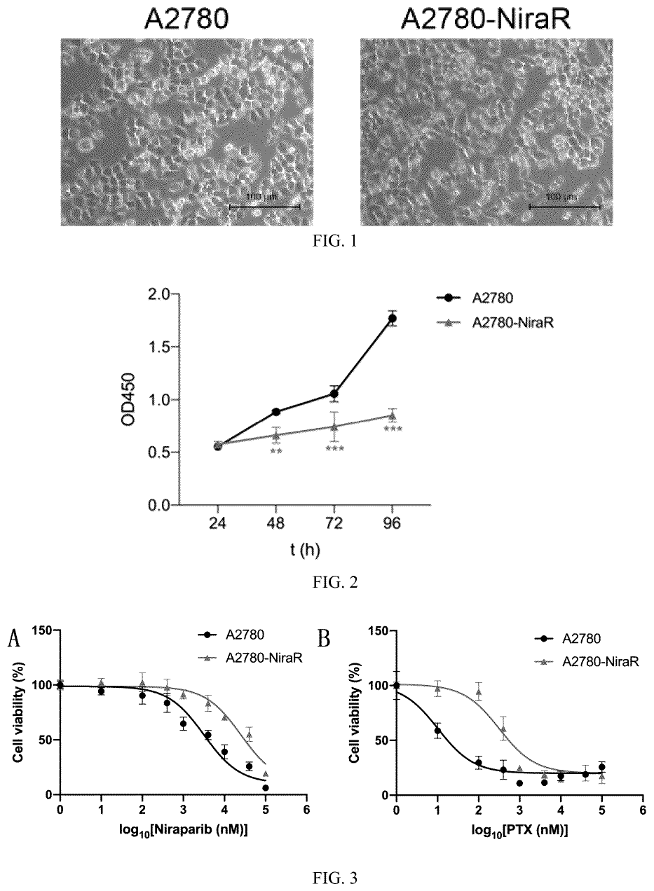

An inverted phase contrast microscope (Leica DMI4000B, Leica, Germany) was used to observe and photograph the cell morphology in logarithmic growth phase. As shown in FIG. 1, the parental A2780 cells were mainly spindle-shaped; A2780-NiraR cells were mainly round, and compared with the original strain, A2780-NiraR cells became round in shape, large in size and poorly differentiated.

2. CCK-8 method was used to determine the cell growth curve.

A CCK-8 kit of Shanghai Beyotime Biotechnology Co., Ltd. was used for the cell proliferation experiment. Cells in logarithmic growth phase were collected, and the concentration of cell suspension was adjusted to 2×10⁴ cells/ml; 100 μl of the cell suspension was added to each well of 96-well plate, and the density of the cells to be tested was 2000 cells/well, 3 replicate wells were set; cultures were incubated overnight in the incubator with 5 vol % CO₂ at 37° C. until the cells were grown as adherent monolayers; OD values were measured at 24 h, 48 h, 72 h and 96 h respectively; 10 μl CCK-8 solution was added to each well; after the 96-well plate was incubated in a cell incubator for 2 h in the dark, the absorbance was measured at 450 nm by a microplate reader. Each experiment was repeated for 3 times, and the growth curves of cells were drawn. As shown in FIG. 2, it can be seen that the proliferation rate of A2780-NiraR cells slowed down and the doubling time increased.

3. Determination of resistance index

A cell counting kit-8 (CCK-8) of Shanghai Beyotime Biotechnology Co., Ltd. (C0038) was used in the cytotoxicity experiment. Cells in logarithmic growth phase were collected, and the concentration of cell suspension was adjusted to 5×10⁴ cells/ml; 100 μl of the cell suspension was added to each well of 96-well plate, and the density of the cells to be tested was 5000 cells/well; cultures were incubated overnight in the incubator with 5 vol % CO₂ at 37° C. until the cells were grown as adherent monolayers, and then drugs with concentrations of 1×10⁻³-100 μM were added respectively; 3 replicate wells were set for each concentration; OD values were determined after 72 h of drug treatment; 10 μl CCK-8 solution was added to each well; after further incubation in the cell incubator for 2 h, the absorbance was measured at 450 nm using a microplate reader (Bio-Rad, Model 680), as shown in FIG. 3. The IC50 of the drug was calculated.

Resistance index (RI)=IC50 of drug-resistant cells/IC50 of parental cells.

The analysis showed that the resistance index (RI) to Niraparib of A2780-NiraR cells was 8.274, which was highly resistant, while the resistance index (RI) to Taxol was 31.02. See Table 1 for details.

TABLE 1

| | A2780 | A2780-NiraR | Drug resistance index |
|---|---|---|---|
| IC₅₀Paclitaxel (μM) | 0.010 | 0.318 | 31.02 |
| IC₅₀Niraparib (μM) | 3.166 | 26.19 | 8.274 |

4. Stability detection of drug-resistant cells

After the establishment of A2780-NiraR cell strain, the drug resistance was re-tested after cell resuscitation following 3-month drug-withdrawal culture and 6-month liquid nitrogen cryopreservation. The procedure was the same as that of the resistance index determination in part 3. The results showed that the IC50 of A2780-NiraR was 24.02 UM and the stability was 91.7%.

To sum up, A2780-NiraR cells of the present application were round in shape and large in volume; the proliferation rate slowed down significantly, and the doubling time increased significantly. The resistance index to Niraparib was 8.274. In addition to resistance to Niraparib, A2780-NiraR cells were cross-resistant to Taxol. Moreover, after the establishment of A2780-NiraR cell strain, the drug resistance was re-tested as more than 90% of original after cell resuscitation following 3-month drug-withdrawal culture and 6-month liquid nitrogen cryopreservation, showing an excellent drug resistance stability. The present application provided a cell model for studying the drug resistance mechanisms of ovarian cancer, analyzing the susceptibility of chemotherapeutic drugs, screening chemotherapeutic drugs, and researching more effective tumor treatment methods.

What is claimed is:

1. A human ovarian cancer Niraparib-resistant cell strain named A2780-NiraR, preserving in China Center for Type Culture Collection with a preservation number of CCTCC NO: C202299.

2. The cell strain according to claim 1, wherein a resistance index of A2780-NiraR cell strain to Niraparib is 8.274.

3. The cell strain according to claim 1, wherein A2780-NiraR cell strain has a drug resistance of 91.7% of original after 3-month drug-withdrawal culture, 6-month liquid nitrogen cryopreservation and resuscitation following establishment.

4. Progeny cells of the human ovarian cancer Niraparib-resistant cell strain according to claim 1.

5. Use of the human ovarian cancer Niraparib-resistant cell strain according to claim 1, comprising:
(1) study of tumor drug resistance mechanisms in vitro;
(2) analysis of chemotherapeutic drug susceptibility in vitro;
(3) preparation of a tumor cell model or a tumor animal model;
(4) screening and evaluation of chemotherapeutic drugs in vitro;
(5) development of drug resistance reversal agents in vitro.
